(19) United States
(12) Plant Patent Application Publication
(10) Pub. No.: US 2003/0237121 P1
(43) Pub. Date: Dec. 25, 2003

(54) Mandarin hybrid tree named "TDE3"

(75) Inventors: Mikeal L. Roose, Riverside, CA (US); Timothy A. Williams, Riverside, CA (US); Robert K. Soost, Inverness, CA (US); James W. Cameron, Salem, OR (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Regents of the University of California Office of Technology Transfer University of California, Oakland, CA

(21) Appl. No.: 10/177,479

(22) Filed: Jun. 20, 2002

(51) Int. Cl. A01H 5/00

(52) U.S. Cl. PLT/202

(57) ABSTRACT

A new mandarin hybrid called “TDE3” is distinguished by production of fruit that combine mid-season maturity, medium large fruit size, attractive deep orange rind color and virtual absence of seeds with rich fruit flavor.

### BACKGROUND OF THE INVENTION

The pedigree of TDE3 is shown in FIG. 1. In 1973, pollen from Encore mandarin was applied to stigmas of a tetraploid (Temple×4N Dancy) hybrid and the pollinated flowers were bagged to prevent insect pollination. Fruits were collected in winter 1974, seeds extracted from each fruit, and each seed was planted. The chromosome number of each seedling was determined and those identified as triploid seedlings were budded onto Troyer rootstock. The resulting trees were planted in the field in Riverside, Calif. in 1976. These trees were evaluated for tree vigor, bearing, and seediness, fruit flavor, fruit color, and other fruit quality traits from bearing until 1985. Five trees were selected from the original population and repropagated by budding onto C-32 citrange, C-35 citrange, Troyer citrange, and trifoliate orange rootstocks. Two trees of the selection now called TDE3 selection were planting in the field in Riverside in 1987. When they began fruiting (approximately in 1990), these trees were evaluated for the same tree and fruit quality traits as the original trees. In 1987, the selection now called TDE3 was chosen for additional testing because it combined medium or large fruit size, low seed number, rich fruit flavor, deep orange rind and flesh color, and acceptable peelability. Budwood of this selection was tested for viruses and other pathogens by the Citrus Clonal Protection Program and virus-free bud source trees were planted at Lindcove Research and Extension Center, Exeter, Calif. in 1991.

Using this virus-free budwood source, additional trees were propagated and planted at several California locations between 1993 and 1996. These included one location in the Coachella Valley (the Coachella Valley Agricultural Research Station-CVARS, 8 trees), Ojai (12 trees) and Santa Paula (4 trees) in Ventura Co., Valley Center (San Diego Co., 10 trees), and two locations in the San Joaquin Valley, (Lindcove Research and Extension Center, 8 trees and Orange Cove, 8 trees). These trial plantings provide most of the available data on TDE3. Several different rootstocks have been used in these evaluations, mostly Carrizo citrange, C35 citrange, Rich 16-6 trifoliate, Cleopatra mandarin, and Schaub rough lemon. The trees in Valley Center are topworked Valencia orange on Troyer citrange rootstock. In general, no major effects of these rootstocks on fruit quality of TDE3 were observed and no incompatibilities have been evident, but longevity of trees on various rootstocks is not known.

### BRIEF SUMMARY OF THE INVENTION

The present invention provides a novel mandarin hybrid having the characteristics described and illustrated herein. The hybrid TDE3 produces fruit that combines mid-season maturity, medium large fruit size, attractive deep orange rind color and virtual absence of seeds with rich fruit flavor.

### BRIEF DESCRIPTION OF THE DRAWINGS

FIG. 1 illustrates the pedigree of TDE3. All cultivars are C. reticulata except orange, which is C. sinensis.

FIG. 2 illustrates, clockwise from top left: a nine-year-old tree of TDE3 on Carrizo rootstock; fruit on tree; branching pattern; flower buds; flowers; leaves; and shoots.

FIG. 3 illustrates fruit of TDE3 sampled from nine-year-old tree on Carrizo rootstock.

FIGS. 4A-F illustrate the solids:acid ratio of TDE3 at six California locations over five years. Points plotted are means of all samples collected on a given date. Solid lines connect means for sampling dates within the same season. The dashed line is a liner regression of solids:acid on sampling date using data from all years. The regression equation and r² value are shown in each figure.

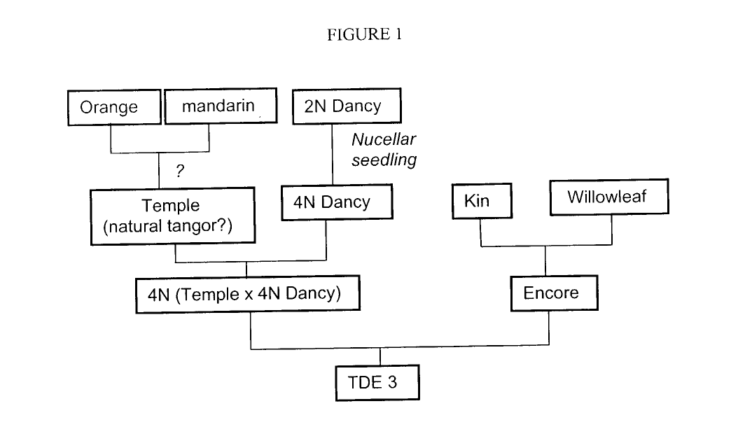

### DETAILED DESCRIPTION OF THE INVENTION

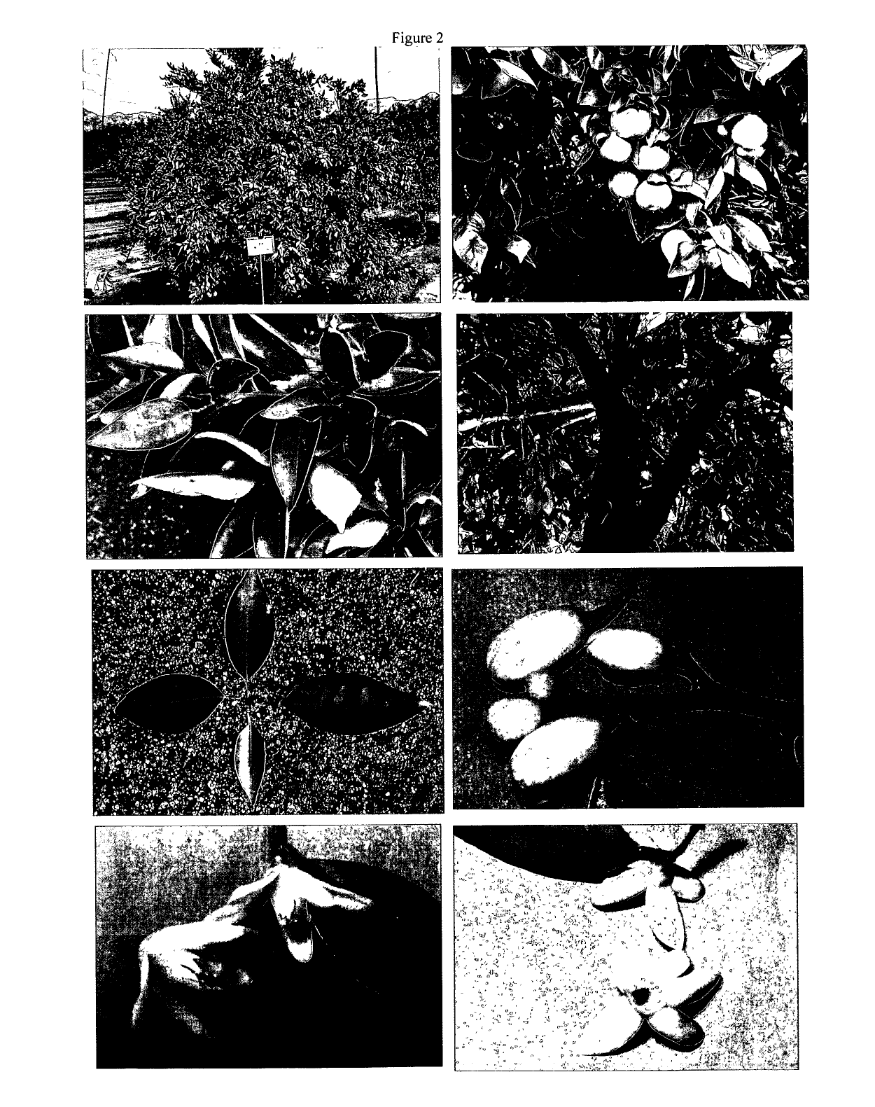

Tree shape (see FIG. 2) is approximately sphereoid, rather similar to that of orange trees. The trees have not been noted as particularly susceptible to any diseases and, based on a freeze in 1999, appeared only slightly more cold hardy than oranges of similar age. Leaves are simple, brevipetiolate, lanceolate, with entire or slightly sinuate margins (FIG. 2). The petiole shape is narrow and linear in shape. n comparison with most old-line citrus cultivars, trees of TDE3 are fairly thorny, with normal branches having short length (6 mm) thorns at about 13% of the nodes, and vigorous sprouts having long (27 mm) thorns at about 63% of nodes. Thorniness will probably decrease as the cultivar ages.

Flowers of TDE3 are typically hermaphroditic, with white petals and yellow anthers (FIG. 2). Trees flower from early April into May at most locations. Pollen is somewhat sparse, with 11% viability estimated in an in vitro germination test. Pollen tube growth is also less vigorous than that of other mandarins with high fertility. Many fruit are born inside the canopy, which limits sunburn.

If sufficient fruit was available, 10-fruit samples were collected from each location two or three times each year beginning in 1997 or 1998. Generally samples were collected from two or three trees on each sampling date. These fruit were evaluated in Riverside for a range of traits as summarized in Table 1.

TABLE 1
Fruit characteristics of TDE3 averaged over 6 locations
and 4 seasons. Samples were collected from November to
April or May. “N” indicates the total number of
fruit samples analyzed. Data are average over trees
on various rootstocks.

| Trait | N | Mm | Max | Mean | SD |
|---|---|---|---|---|---|
| Fruit height (mm) | 286 | 42.0 | 80.4 | 56.2 | 6.96 |
| Fruit width (mm) | 287 | 50.0 | 99.7 | 66.0 | 8.07 |
| Fruit height:width | 286 | 0.64 | 1.00 | 0.85 | 0.054 |
| Rind cobra | 287 | 4.0 | 13.0 | 11.9 | 2.22 |
| Rind texture^{b} | 287 | 2.8 | 6.5 | 3.7 | 0.63 |
| Neck^{c} | 287 | 0 | 2.50 | 0.79 | 0.673 |
| Peelability^{d} | 287 | 5.00 | 10.00 | 8.20 | 0.953 |
| Rind thickness (mm) | 287 | 2.50 | 6.00 | 3 86 | 0.725 |
| Seeds per fruit | 287 | 0 | 5.00 | 0.29 | 0.580 |
| Fruit weight (g) | 283 | 71.5 | 290.5 | 134.4 | 42.93 |
| Juice content (%) | 279 | 22.5 | 72.2 | 47.5 | 7.21 |
| Soluble solids (%) | 279 | 5.50 | 16.90 | 11.99 | 2.184 |
| Acid (%) | 279 | 0.39 | 3.40 | 1.27 | 0.543 |
| Solids:acid | 279 | 3.60 | 26.52 | 10.78 | 3.916 |

^{a}Visua1 rating on a scale of 0-13; 0 = green, 13 = red-orange
^{b}Visual rating on a scale of 1-8; 1 = very smooth, 8 = extremely coarse
^{c}Visual rating on a scale of 0-3; 0 = no trace of neck, 3 = neck with a diameter at least 50% of fruit diameter
^{d}Subjective rating of ease of peeling a single fruit; 1 = very difficult, 10 = a fruit with completely separated rind and segments. Fruit with ratings of 7 or higher would be relatively easy to peel.

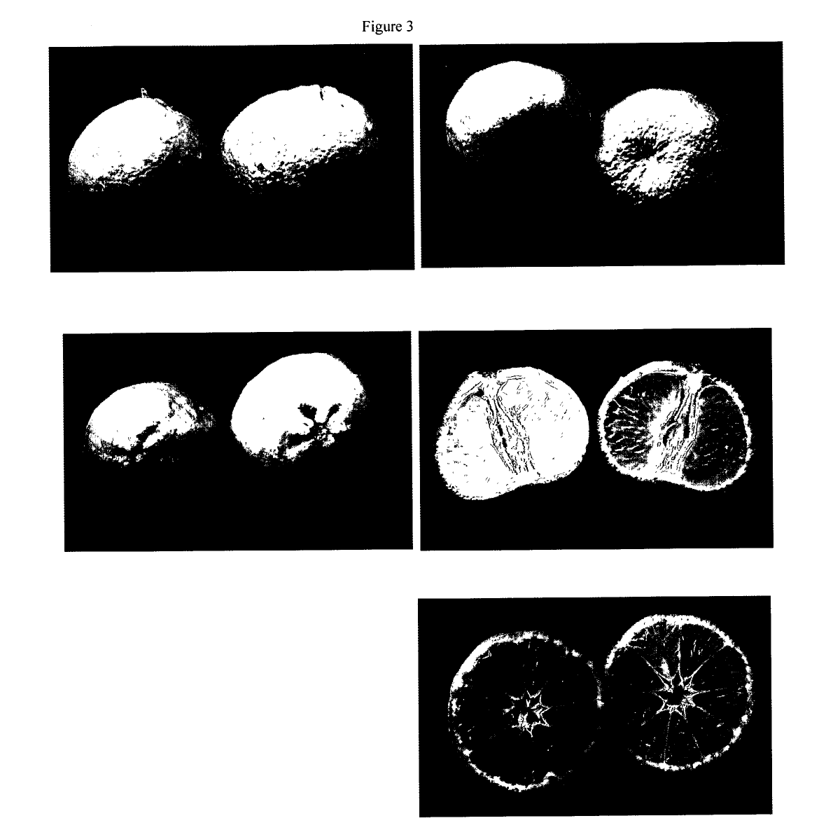

Based on this data, TDE3 fruit are oblate in shape (FIG. 3). The fruit base (stalk end) is convex, with some fruit having a small neck, and the apex (stylar end) is depressed (FIG. 3). The stylar scar is typically small and closed. The average fruit size is medium-large for a mandarin (classed as Jumbo by California state standards). Rind color is deep orange with color scores of L=59.6, C=61.1, H=63.7 for fruit harvested in Riverside on February 10. The rind texture is variable, depending on tree age and crop. For older trees with a moderate to heavy crop, rind texture is papillate, with fairly conspicuous oil glands (FIG. 3). The rind of fruit from trees with very light crops is often excessively rough or bumpy. The rind is of medium thickness and easy to peel when fruit are mature, but can be much more adherent early in the season. The fruit is typically very juicy. Flesh color is deep orange.

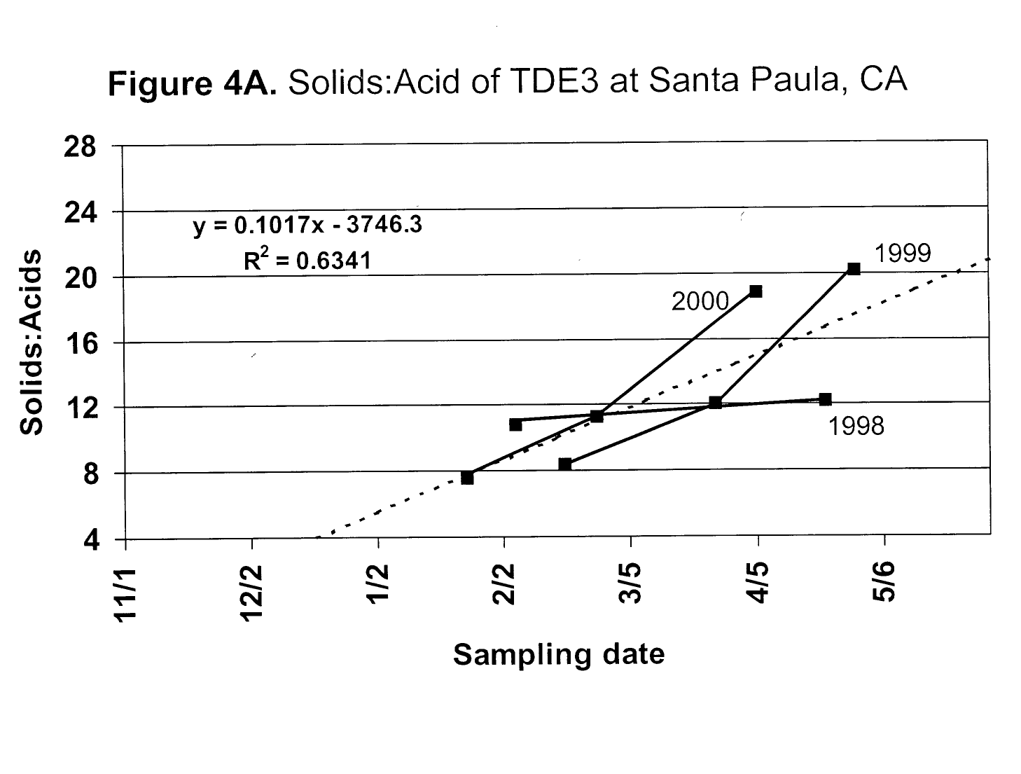

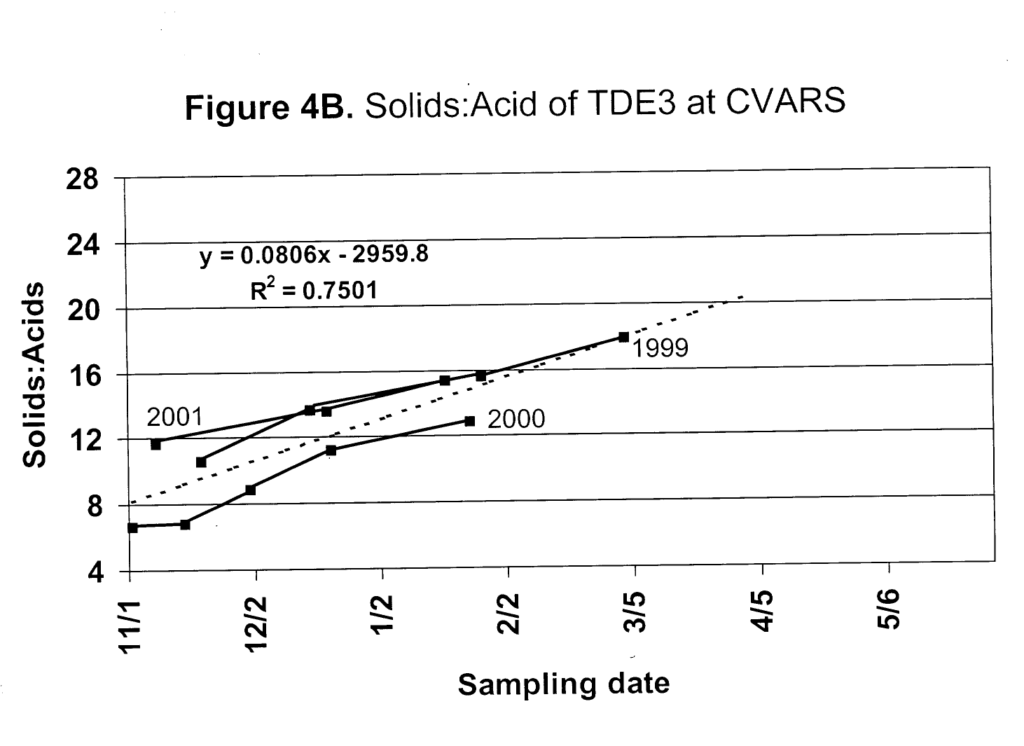

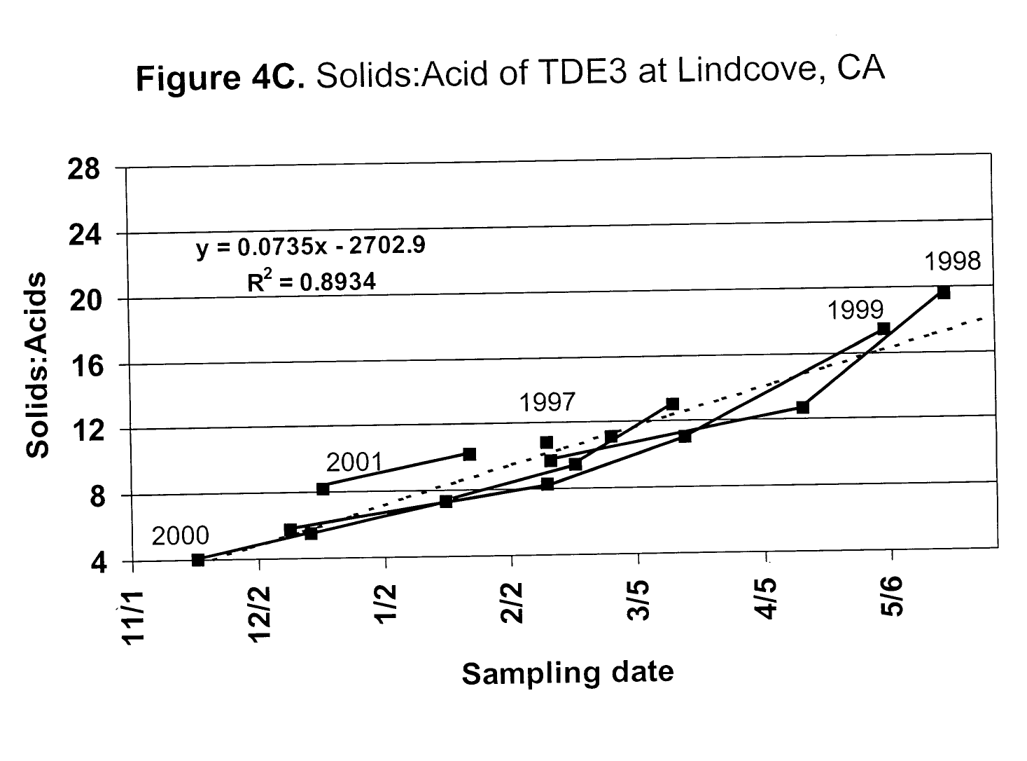

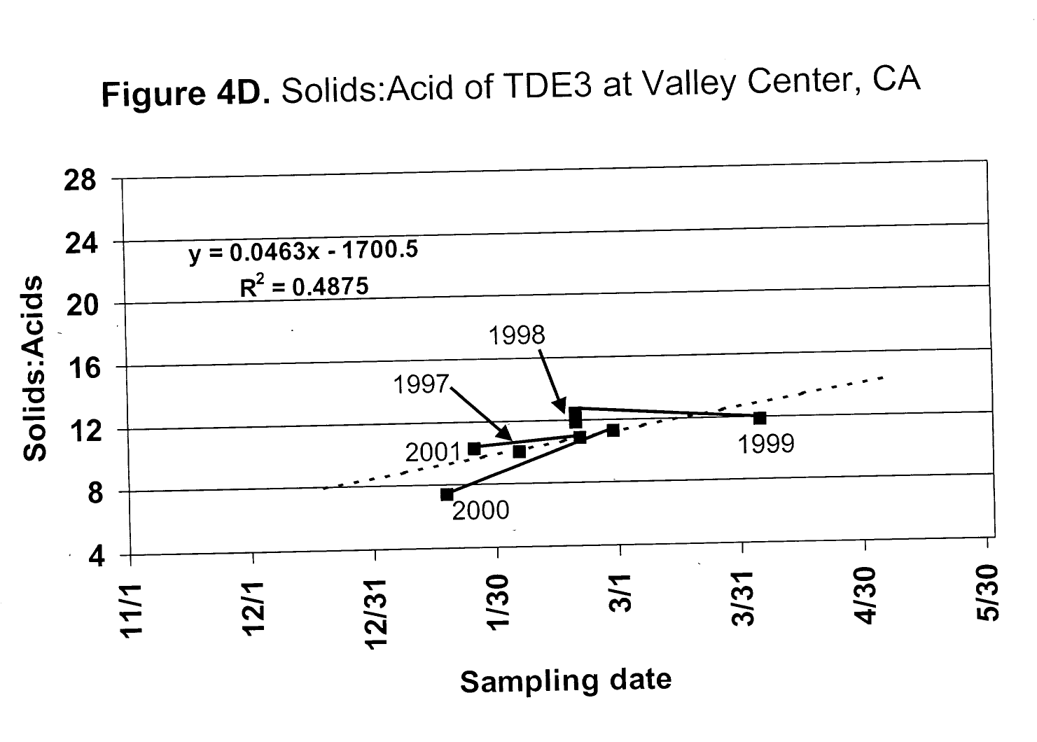

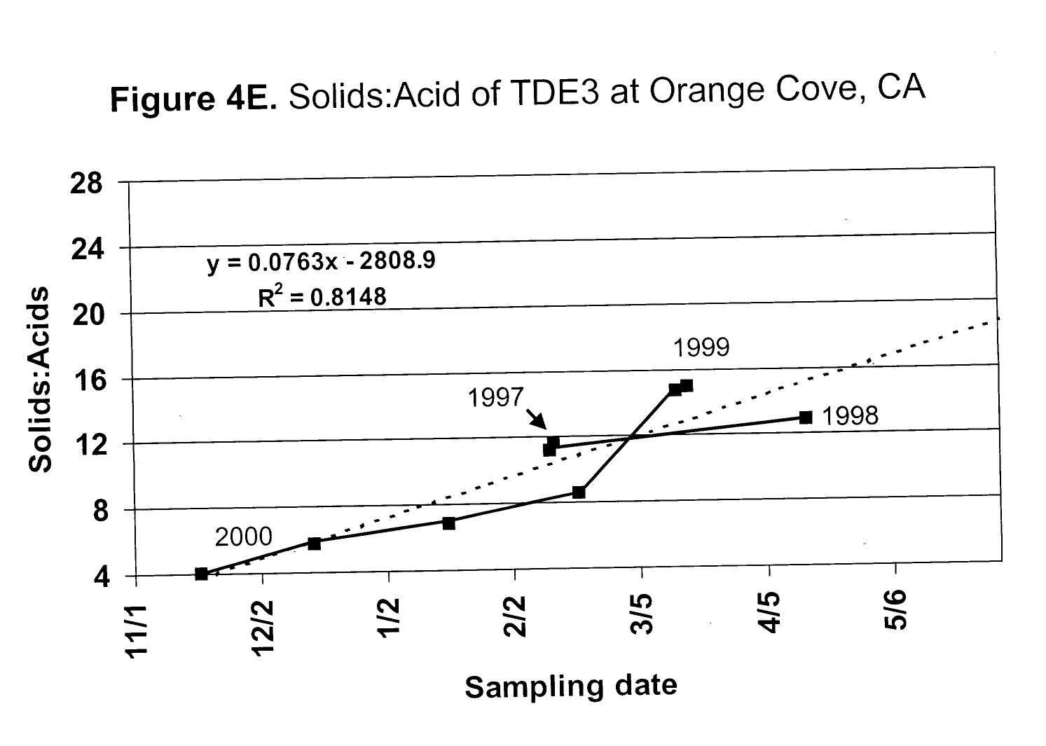

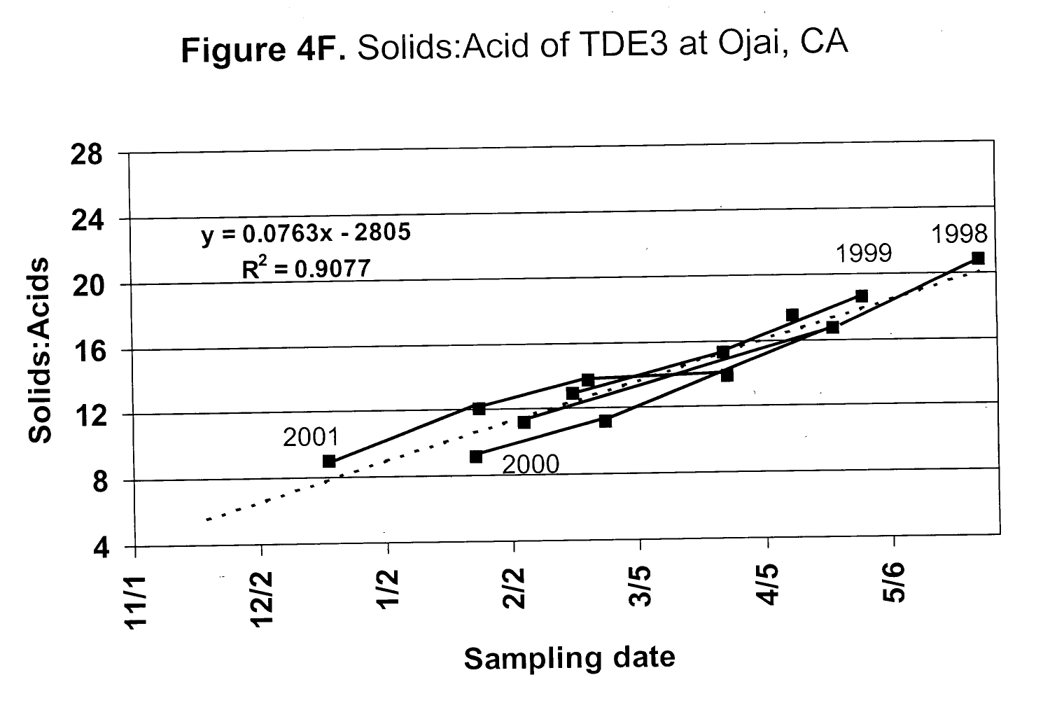

Important determinants of maturity date for citrus fruit are the solids:acid ratio and juice content. Although the overall juice content was high (>40%) in most samples, juice content was negatively correlated with sampling date at all 6 locations, and significantly so at CVARS and Lindcove. Drying of fruit was particularly evident in samples collected after early March at most locations. At CVARS, juice content declined between November and December in all three years for which samples were analyzed. Solids:acids ratio (FIG. 4) was significantly correlated with sampling date at all locations except Valley Center, where the range of sampling dates was somewhat narrower. Using these regressions, the estimated dates on which fruit reached an 8:1 solids:acid ratio was November 1 for CVARS, December 15 for Valley Center and Ojai, January 8 for Orange Cove, January 12 for Lindcove, and January 20 for Santa Paula.

Yield of TDE3 was evaluated from visual ratings of crop relative to tree size at each location from 1998-99 to 2001-2002. The rating scale ranged from 0 (no crop) to 5 (very heavy crop). The topworked trees in Valley Center showed the highest and most consistent crops, ranging between 2 and 4.5 over the 4 years studied. Crops at Ojai were also good, being 1.5 or greater in all years. At Lindcove, Orange Cove, and Santa Paula, crop ratings indicated alternate bearing, with values of about 3, 0.5, and 1.5-4 in the last 3 years. Yield at Lindcove in 2000 and 2001 was 53 and 4.6 kg tree⁻¹, while at Orange Cove it was 63 and 0 kg tree⁻¹.

Trees that were screened to exclude bees during flowering produced very few fruit for two consecutive years, but it is possible that TDE3 is self-fertile but requires pollination for fruit set.

Two siblings of TDE3, “TDE2” and “TDE4,” were compared to TDE3. TDE3 is distinct from these cultivars in having the earliest maturity date, a less oblate shape, and distinctive flavor. The rind color of TDE4 is usually deeper orange than that of TDE3. Trees or fruit of TDE3 can be distinguished from those of other mandarins, including TDE2 and TDE4, using simple sequence repeat (SSR) DNA markers. Using TDE3 DNA as template, PCR primer set CAC15 (F=TAAATCTCCACTCTGCAAAAGC, R=GATAGGAAGCGTCGTAGACCC) amplified a band of 158 bp, while TDE2 and TDE4 both had two bands of 149 and 158 bp. Bands amplified with TAA15 (F=GAAAGGGTTACTTGACCAGGC, R=CTTCCCAGCTGCACAAGC) combined with those amplified with CAC15, TAA33 (F=GGTACTGATAGTACTGCGGCG, R=GCTAATCGCTACGTCTTCGC) and TAA03 (F=AGAGAAGAAACATTTGCGGAGC, R=GAGATGGGACTTGGTTCATCACG) distinguished TDE3 from the following cultivars: Dancy, Temple, Encore, King, Willowleaf, Wilking, Gold Nugget, Pixie, W. Murcott Afourer, Ellendale, Hemandina Clementine, Fortune, Kara, Kinnow, Murcott, Nova, and Ponkan.

During the 1999-2000 and 2000-01 seasons, fruit of TDE3 and various other mandarin varieties were harvested and evaluated by a taste panel. In some cases, evaluations were done before and after storage at two different temperatures. Fruit were rated on a 9 point scale, where a score of 1 is “Dislike extremely”, 5 is “Neither dislike or like”, and 9 is “Like extremely”.

Vigor of TDE3 trees has varied greatly across locations. At CVARS, canopy volumes of 7-year-old trees averaged 21.1 m³. In contrast, at the cooler Santa Paula and Ojai locations, 7-year-old trees averaged 6.2 and 3.6 m³. 7-year-old trees at Lindcove and Orange Cove averaged 9.9 and 7.3 m³. Trees at CVARS have had very small crops relative to tree size, perhaps contributing to greater vegetative growth. Size of the topworked trees in Valley Center has not been measured since they are not comparable to trees in other locations, but in general the topworked trees are quite vigorous. Trees on Schaub rough lemon were larger than those on Carrizo citrange or C35 citrange at Santa Paula and Ojai. At Lindcove and CVARS, trees on Rich 16-6 trifoliate and Cleopatra were smaller than those on Carrizo and C-35 citranges. Fruit from trees on Schaub rough lemon generally have slightly lower solids and acid than those from trees on Carrizo citrange, C35 citrange, or trifoliate orange, but this effect is less noticeable than with oranges. No evidence of stock-scion incompatibilities was evident.

The most comparable mandarin is W. Murcott Afourer, which generally has a smoother rind than TDE3. However, when pollinizers are present, TDE3 has fewer seeds than W. Murcott Afourer. For example, data from a mixed planting in Riverside shows fruit of W. Murcott Afourer average 7.2 seeds per fruit, while those of TDE3 average about 0.5.

Storage and Sensory Evaluation of TDE3 were performed. Fruit were sampled from test plots at Lindcove and Orange Cove, run over a packline at the University of California Lindcove Research and Extension Center and waxed on Jan. 21, 2000. The fruit were stored 11 days at 68° F., 12 days at 37° F. followed by 7 days at 55° F., or 12 days at 41° F. followed by 7 days at 55° F. These samples represent early-season fruit of TDE3, the fruit from Lindcove and Orange Cove having solids:acid ratios of 7.6 and 6.8, respectively. Their ratings were good for all traits before storage, and were slightly changed or improved by both cold storage treatments. Storage at 68° F. reduced scores for visual appeal and peelability.

Overall, in comparison with the other cultivars sampled, later season samples generally were liked for taste or peelability by the panels. Storage tests indicate that TDE3 fruit can be stored at cold temperatures for at least 12 days without greatly affecting visual appeal or taste.

TDE3 can be propagated on many available citrus rootstocks by budding. To reduce thorniness, budwood should be selected from thornless, upper canopy branches. Tree spacing in field plantings will depend on vigor of the rootstock. For Carrizo citrange rootstocks, a recommended tree density is about 150 trees per acre. Higher densities are possible, but will require more frequent pruning or hedging. Care of young trees should be similar to that used for other mandarins or oranges. Trees have performed best in locations with more moderate climates such as the inland valleys of southern California. Trees can be grown with pollinizer cultivars such as Minneola, Valencia orange, or unrelated mandarins (not Temple, Dancy, Encore or other TDE hybrids) that produce viable pollen. Optimal pruning practices have not yet been developed, but in many locations trees will perform well with relatively little pruning. Maturity dates will vary with location, probably depending on the number of heat units and soil conditions.

As in some other mandarins, sprays with gibberellic acid may increase fruit set when pollinizers and/or pollinators are inadequate.

What is claimed is:

1. A new and distinct variety of mandarin hybrid tree having the characteristics described and illustrated herein.
